(19) United States
(12) Patent Application Publication
(10) Pub. No.: US 2024/0237923 A1
(43) Pub. Date: Jul. 18, 2024

(54) Mobility Analysis

(71) Applicant: MiiCare Ltd, London (GB)

(72) Inventor: Kelvin Summoogum, London (GB)

(21) Appl. No.: 18/554,421

(22) PCT Filed: Apr. 7, 2022
(86) PCT No.: PCT/GB2022/050885

§ 371 Date: Oct. 6, 2023

(30) Foreign Application Priority Data
Apr. 8, 2021 (GB) GB2105050.5

(51) Int. Cl. A61B 5/11; A61B 5/00

(52) U.S. Cl. CPC A61B 5/112; A61B 5/1123; A61B 5/4023; A61B 5/486; A61B 5/7264; A61B 2562/0204

(57) ABSTRACT

A method for measuring the mobility of a subject, the method comprising: receiving an audio signal from one or more microphones: for each of a plurality of overlapping regions of the audio signal, classifying the region as containing the sound of a footstep using a first supervised learning algorithm, determining that two or more of the regions classified as containing the sound of a footstep correspond to a series of two or more consecutive footsteps of a subject; and using a first neural network, analysing the determined two or more regions to determine a mobility factor.

### FIELD

This invention relates to a method and system for analysing the mobility of a subject.

### BACKGROUND

Keeping elderly people in the safe environment of their home for as long as possible is a challenge of increasing urgency because of the growing and ageing population and budgetary pressures for residential care costs in many countries. Indeed, the prolongation of life expectancy in modern society has led to an increased ageing population, with many elderly people living with age-related pathologies like dementia. People living with dementia (PLWD) are more prone to falls than cognitive-intact people, falling more often and suffering fractures and muscle tone loss that result in high rates of morbidity, mortality and hospitalisation. Addressing the unmet need of mobility monitoring and fall prevention in dementia care is a matter of high priority that can lessen financial pressure on local authorities.

New advances in remote sensing using video image processing have made non-contact monitoring possible. It is even possible to record the blood volume changes associated with the cardiac cycle remotely from facial images. However, these systems require a video camera placed in a room approximately 2 m away from a human subject. Home vision-based monitoring can also be effective in mobility and gait measurement, to predict future falls in older adults, or to monitor activities of daily living (ADL). While these non-contact monitoring methods yield huge benefits there is significant reluctance among the elderly adults and their families for their homes to be fitted with closed-circuit television (CCTV) cameras. Infrared motion sensor detection is another effective non-contact method for activity detection in home settings. These basic sensors, however, can only acquire limited information, mainly of the subject's movement, without being able to provide useful indicators of likelihood of a fall or changes in mobility or gait.

Several assisted-living technologies, including fall detection, exist in the market. Although efficient to detect a fall, these devices raise significant ethical issues, especially in view of liberty and privacy. In addition, none of the existing technologies are able to reliably predict falls and are therefore not capable of preventing them from happening.

Sound Event Detection (SED) (also referred to as Acoustic Event Analysis (AEA)) is widely researched for applications such as smart home automation and voice activated systems in homes and cars. However, traditional AEA methods tend to perform poorly where there is interfering acoustic noise, such as in a home setting.

There is a need for an improved method of monitoring the mobility of a subject.

### SUMMARY OF THE INVENTION

According to the present invention there is provided a method for measuring the mobility of a subject, the method comprising:

receiving an audio signal from one or more microphones;

for each of a plurality of overlapping regions of the audio signal, classifying the region as containing the sound of a footstep using a first supervised learning algorithm;

determining that two or more of the regions classified as containing the sound of a footstep correspond to a series of two or more consecutive footsteps of a subject; and

using a first neural network, analysing the determined two or more regions to determine a mobility factor.

Analysing the determined two or more regions to determine a mobility factor using a first neural network may comprise analysing the determined two or more regions to determine one or more of a cadence of the series of footsteps, a hesitancy of the subject, and a balance of the subject, and wherein the mobility factor is determined in dependence on one or more of the cadence, hesitancy, and balance.

The first supervised learning algorithm may comprise a support vector machine classifier.

Classifying a region as containing the sound of a footstep using the first supervised learning algorithm may comprises: analysing the region to locate one or more markers indicative of footstep events; and, if the region contains more than a predefined threshold number of footstep events, classifying that region as containing the sound of a footstep.

Classifying a region as containing the sound of a footstep using the first supervised learning algorithm may comprise deriving a spectral energy of the region of the audio signal, wherein analysing the region to locate one or more markers indicative of footstep events comprises analysing the spectral energy of the region of the audio signal to locate one or more markers indicative of footstep events.

Classifying a region as containing the sound of a footstep using a first supervised learning algorithm may comprise: determining one or both of the mean and the variance of the spectral energy in the region of the audio signal; determining whether determined mean and/or variance of the spectral energy falls within a predefined range of an expected mean and/or variance respectively.

A footstep event may comprise one or more of: a heel strike, a tiptoe collision, and a toe scrape.

The method may further comprise training the supervised learning algorithm.

The method may further comprise providing feedback indicative of the mobility factor to one or more users.

The feedback may comprise one or more of visual, audible, and tactile feedback.

Providing feedback may comprise providing an alert to one or more of the subject, a medical professional, and a designated person if the mobility factor changes by more than a predetermined threshold amount.

The method may further comprise determining that a region classified as containing the sound of a footstep contains the sound of a footstep of the subject by applying an unsupervised learning algorithm trained to identify the sound of the subject's footsteps.

The unsupervised learning algorithm may comprise a Gaussian mixture model.

The method may further comprise training the unsupervised learning algorithm to identify the sound of the subject's footsteps.

The audio signal may be received from two or more microphones.

The method may further comprise capturing that part of the audio signal received by a first microphone and validating the captured audio signal by comparing the captured audio signal with that part of the audio signal received by a second microphone.

The method may further comprise comparing the time that a feature in the audio signal is received by a first microphone with the time that a feature in the audio signal is received by a second microphone to determine a region of space from which the feature in the audio signal originated.

There is also provided a system for measuring the mobility of a subject, comprising: a footstep detection unit configured to receive an audio signal from one or more microphones and, for each of a plurality of overlapping regions of the audio signal, classify the region as containing the sound of a footstep using a first supervised learning algorithm; and a footstep analysis unit configured to determine that two or more of the regions classified as containing the sound of a footstep correspond to a series of two or more consecutive footsteps of a subject and to, using a first neural network, analyse the determined two or more regions to determine a mobility factor.

The footstep analysis unit may be configured to analysing the determined two or more regions to determine a mobility factor using a first neural network by analysing the determined two or more regions to determine one or more of a cadence of the series of footsteps, a hesitancy of the subject, and a balance of the subject, and wherein the mobility factor is determined in dependence on one or more of the cadence, hesitancy, and balance.

The first supervised learning algorithm may comprise a support vector machine classifier.

The footstep detection unit may be configured to classify a region as containing the sound of a footstep using the first supervised learning algorithm by: analysing the region to locate one or more markers indicative of footstep events; and if the region contains more than a predefined threshold number of footstep events, classifying that region as containing the sound of a footstep.

The footstep detection unit may be configured to classify a region as containing the sound of a footstep using the first supervised learning algorithm by: deriving a spectral energy of the region of the audio signal, wherein analysing the region to locate one or more markers indicative of footstep events comprises analysing the spectral energy of the region of the audio signal to locate one or more markers indicative of footstep events.

The system may further comprise a feedback unit configured to provide feedback indicative of the mobility factor to one or more users.

The feedback unit may be configured to provide one or more of visual, audible, and tactile feedback

There is also provided a computer readable storage medium having stored thereon computer readable instructions that, when executed at a computer system, cause the computer system to perform the method of any of claims 18 to 24.

There may be provided computer program code for performing a method as described herein. There may be provided non-transitory computer readable storage medium having stored thereon computer readable instructions that, when executed at a computer system, cause the computer system to perform the methods as described herein.

### DESCRIPTION OF THE DRAWINGS

The present invention will now be described by way of example with reference to the accompanying drawings. In the drawings:

FIG. 1 shows a schematic diagram of the parts of the human foot that impact the ground during a footstep.

FIG. 2 shows the motion of parts of the foot throughout a footstep.

FIG. 3 shows an exemplary sound waveform caused by a footstep.

FIG. 4 shows a schematic diagram of an exemplary system for analysing the mobility of a subject.

FIG. 5 shows an exemplary plot of spectral energy against time for a normally walking subject.

FIG. 6 shows an exemplary plot of spectral energy against time for a limping subject.

FIG. 7 shows an exemplary diagram of a simple convolutional neural network.

FIG. 8 shows the steps of an exemplary method for analysing the mobility of a subject.

### DETAILED DESCRIPTION

The following description is presented to enable any person skilled in the art to make and use the invention and is provided in the context of a particular application. Various modifications to the disclosed embodiments will be readily apparent to those skilled in the art.

The general principles defined herein may be applied to other embodiments and applications without departing from the spirit and scope of the present invention. Thus, the present invention is not intended to be limited to the embodiments shown but is to be accorded the widest scope consistent with the principles and features disclosed herein.

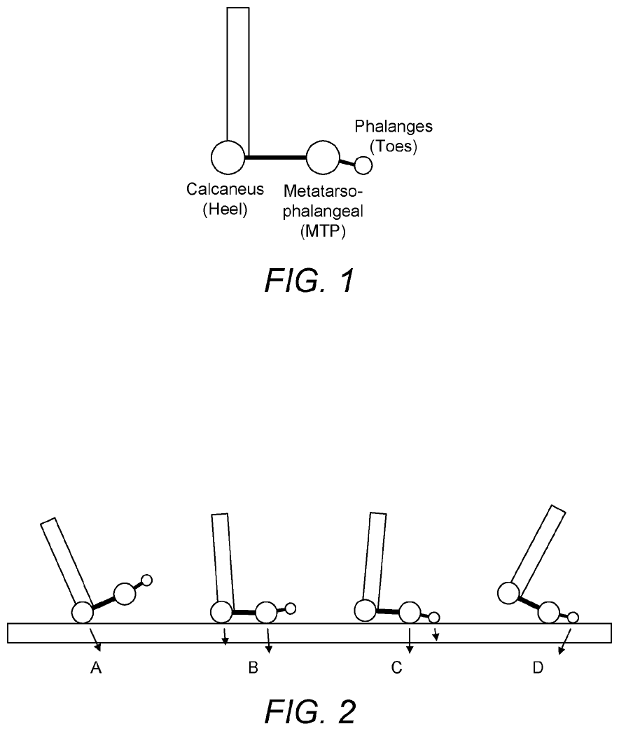

The sound generated by footsteps is linked with the major anatomical components of a human foot. These are depicted in FIG. 1 and include the calcaneus (the heel), the mid foot (including the metatarsophalangeal (MTP) joints), and the phalanges (the toes).

FIG. 2 demonstrates the progression of the location of these parts of the foot in relation to the ground throughout a footstep. At (A), the heel strikes the ground but the MTP and the toes are yet to impact the ground. At (B) the MTP strikes the ground while the heel remains in contact with the ground and the toes are yet to impact the ground. At (C), the toes strike the ground while the MTP remains in contact with the ground. At approximately the same time, the heel lifts off from the ground. At (D), the MTP lifts off from the ground while the toes remain in contact with the ground. In a standard footstep, the toes will be the final part of the foot in contact with the ground. These events (A) to (D) may be referred to as footstep events.

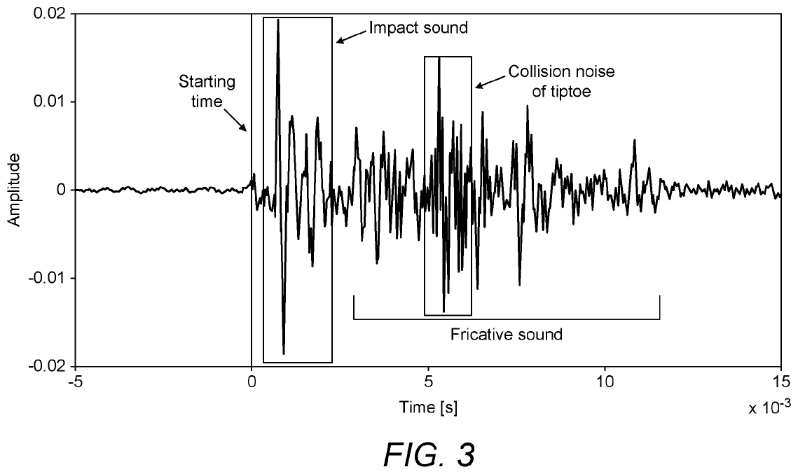

The progression of a footstep as described above generates sound that varies in both in amplitude and frequency as footstep progresses from (A) to (D). FIG. 3 shows an exemplary waveform of a footstep, showing the variation in sound amplitude over time as the footstep progresses. At around t=0 ms, the footstep initiates, corresponding to (A) in FIG. 2. An initial increase in amplitude which decays away is characteristic of the heel striking the ground. Starting at around t=5 ms, the tiptoes collide with the ground, corresponding to (C) in FIG. 2. An initial increase in amplitude, less than that of the heel strike, which then decays away is characteristic of the tiptoes colliding with the ground. The coming together and separation between the foot and the ground produces a baseline of fricative sound throughout the footstep.

These features can be used to distinguish the sound of footsteps from other sounds. Additional features of the waveform can be derived that are also characteristic of a footstep. For example, the time difference between any two of the heel strike peak, the toe strike peak, the starting time, and the ending time may be determined. As a further example, the product, sum, or ratio of the amplitudes of the heel strike peak and the toe strike peak may be determined. As a further example, statistical properties of the waveform may be determined, including one or more of the mean and the variance of the amplitude over the course of the footstep.

While the amplitude of the waveform will be modulated by the mechanical characteristics of footwear and the ground, the time difference between the events will be less affected.

The aforementioned characteristics can also be used to predict the physio-anatomical status of a person. Abnormal gaits like the hemiplegic, diplegic or parkinsonian gait differ significantly from that of a healthy person and are unique in their gait cycle and correspondingly in their gait sound recordings. A cautious gait has been seen as a recurring indicator of initial phases of dementia and in AD patients with mild dementia. Gait parameters like stride length, stride time, stance time and stride velocity have shown to have more variation when a person is in the later stages of dementia than when in the earlier mild stages. “Poor” gait performance has been established as a strong predictor of dementia, particularly in people without Alzheimer's as an existing condition.

The waveform feature variations can also be used to differentiate between footsteps in a group of two or more. Height, weight, muscle mass, femur length, etc all contribute to some or all of these variations. A person with more body weight will generate gait audio with larger amplitude, higher spectral energy at lower frequency bands and more prominent impact peaks in the waveform than those with less weight. Variations in one or more such variables over time can indicate changes in body weight, as an inverse relationship can be established.

The inventor has realised that improved methods of monitoring gait and mobility is possible based on an improved analysis of one or more footsteps of a human subject. Herein, the terms mobility and gait will be used interchangeably.

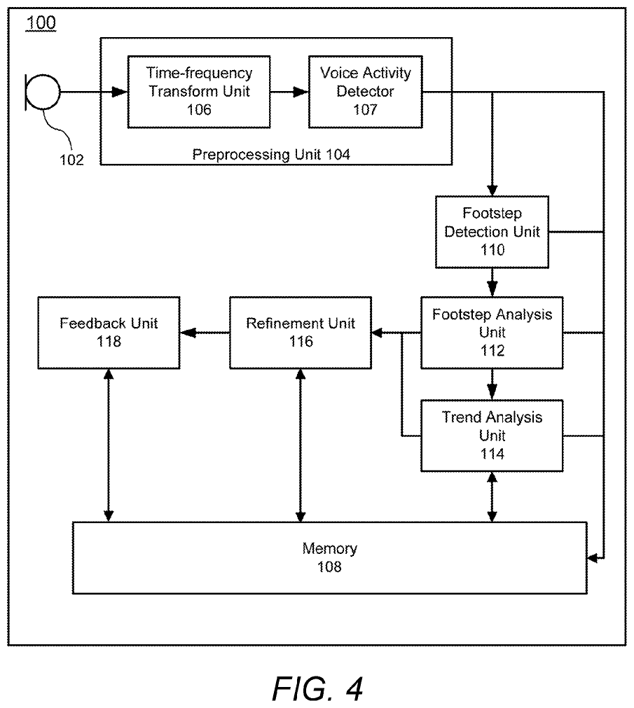

FIG. 4 shows an exemplary system 100 for performing mobility analysis. The system 100 comprises one or more microphones 102 configured to receive an audio signal. The system 100 will preferably be located in an area where the microphone 102 is able to detect an audio signal that includes the sounds of footsteps of a subject, for example in a home, a care home, or a hospital. The system 100 may comprise a plurality of microphones. Using a plurality of microphones may provide several advantages. Firstly, the microphones may be used to performing triangulation and ranging, wherein the potential location of the source of a sound can be determined or at least the range of potential locations can be narrowed. Secondly, one microphone may be used for recording and another microphone may be used for validation, such that if the two microphones detect significantly different signals, the signal can be discarded for a period of time, and not passed to the downstream units for further analysis. Thirdly, providing more than one microphone simply provides additional redundancy in the system, allowing another microphone to be used if one microphone malfunctions.

The system 100 may comprise a preprocessing unit 104. The preprocessing unit 104 may be configured to amplify the received audio signal. The preprocessing unit 104 may be configured to isolate a range of frequencies of the received audio signal for further analysis. This may be referred to as spectrally gating a signal. For example, the preprocessing unit 104 may be configured to isolate the lower end of the frequency spectrum. More specifically, the preprocessing unit 104 may be configured to isolate the frequency spectrum in the range of 10 Hz to 300 Hz. This range of frequencies has been observed to contain majority of the spectral energy content associated with human footsteps. The pre-processing unit 104 may comprise one or more of: a high-pass filter, a low-pass filer, a non-linear filter, and an algorithm to spectrally gate the received audio signal. Since footsteps have major spectral presence between 10 Hz to 300 Hz, the primary operating sampling frequency of microphone(s) 102 may be 16 KHz. The maximum permissible Nyquist frequency of the microphone(s) 102 may be 8 KHz for all acoustic events captured at a 16 KHz sampling frequency.

The preprocessing unit 104 may comprise a time-frequency transform unit 106 configured to convert the received audio signal to the time-frequency domain. The time-frequency transform unit 106 may also be configured to convert the received audio signal from analogue to digital. Converting the received audio signal to the time-frequency domain may comprise performing a Fourier transform on the received audio signal.

The preprocessing unit 104 may comprise a voice activity detector (VAD) 107. VAD 107 is configured to detect the presence of voice in the received audio signal. The system 100 may be configured to store data in memory a 108 in dependence on whether voice is detected in the received audio signal. For example, while the received audio signal contains a voice, the preprocessing unit 104 may be configured not to store the received audio signal in memory 108. This provides additional privacy and data protection for users, particularly when system 100 is installed in a home.

The preprocessing unit 104 may be configured to normalize the volume of the incoming audio signals according to the presence of the footsteps. The preprocessing unit 104 may be configured to perform cleaning and/or suppression of undesirable acoustic events (such as sounds of the television, electronic devices, pet sounds, etc. and privacy-violating acoustic events (such as human speech and conversations). Preprocessing unit 104 may be configured to perform contextual acoustic cleansing, reduction, and/or suppression of the received audio signal in dependence on whether the received audio signal contains undesirable and/or privacy violating acoustic events. This allows the downstream hardware to focus on extracting gait information with the assumption that other irrelevant acoustic event information in the audio signal is negligible. Cleaning/Suppression of the aforementioned undesirable and privacy violating acoustic events may be performed in the frequency domain by using polyphonic multi-class acoustic event classification with a combination of noise reduction techniques like Convolutional Autoencoders, Adaptive Signal Filtering techniques or a combination of both Deep Neural Network Variants and Traditional Signal Filtering.

The system 100 may comprise the memory 108 in which data can be stored. For example, after preprocessing, the received audio signal may be stored in memory 108.

The system comprises a footstep detection unit 110 configured to classify regions of the received audio signal as containing the sound of a footstep using a supervised learning algorithm. The footstep detection unit 110 is configured to do so for each of a plurality of overlapping regions of the received audio signal. The footstep detection unit 110 may comprise a buffer configured to store a region of the received audio signal. Once the footstep detection unit 110 has classified a stored region, the subsequent region may be loaded into the buffer. The regions may comprise 1-2 s of the received audio signal. Each region may be delayed by 0.5-1 s compared to the preceding region. The footstep detection unit 110 may be configured to derive a spectral energy signal for each region by summing the total energy observed across the frequency spectrum for every time instant.

The footstep detection unit 110 may be configured to classify the regions by analysing the region to locate one or more markers indicative of footstep events and, if the region contains more than a predefined threshold number of footstep events, classifying that region as containing the sound of a footstep. The markers may be indicative of footstep events. Analysing the region to locate one or more markers indicative of footstep events may comprise analysing the spectral energy of the region of the audio signal to locate one or more markers indicative of footstep events. The footstep events may comprise one or more of those listed above, for example a heel strike, a toe strike, the starting time, and the ending time.

The footstep detection unit 110 may be configured to classify a region as containing the sound of a footstep by determining one or both of the mean and the variance of the spectral energy in the region of the audio signal and providing the determined mean and/or variance to the supervised learning algorithm. Other measures of the spread of a signal may be used as an alternative to the variance. The footstep detection unit 110 may be configured to determine whether a determined mean and/or variance falls within a predefined range of an expected mean and/or variance respectively. If the mean and/or variance falls within the predefined range, the region may be classified as containing a footstep. If the mean and/or variance falls within the predefined range, the region may be classified as not containing a footstep.

The supervised learning algorithm may comprise a support vector machine (SVM) classifier. Recent studies have been conducted on various feature extraction techniques like Fourier Transform (FT), Homomorphic Cepstral Coefficients (HCC), Short-Time Fourier Transform (STFT), Fast Wavelet Transform (FWT), Continuous Wavelet Transform (CWT), Mel-Frequency Cepstral Coefficients (MFCCs) and Linear Predictive Coding (LPC). Among those mentioned, STFT and MFCCs are the most frequently used. However, the performance of MFCCs degrades significantly when the signal contains noise, overlapping acoustic events and a flat spectrum. The present approach of using an SVM is simpler and more interpretable. Furthermore, by detecting individual footsteps, further information about mobility can be determined through further analysis, as will be discussed below.

The supervised learning algorithm may be trained by providing it with training data for which has been classified by other means (e.g. manually). The training data may comprise the sounds of footsteps as well as other, unrelated sound events including speech and ambient noise. The accuracy of the algorithm in detecting footsteps may then be evaluated. A footstep detection unit 110 trained in such a way was able to achieve an accuracy of 96% when identifying footsteps among 227 various sound events.

The system 100 comprises a footstep analysis unit 112. The footstep analysis unit 112 may receive the output of the footstep detection unit 110, either directly or via memory 108. The footstep analysis unit 112 is configured to determine that two or more of the regions classified as containing the sound of a footstep correspond to a series of two or more consecutive footsteps of a subject. The footstep analysis unit 112 is configured to analyse the determined two or more regions to determine a mobility factor using a first neural network. This meta-analysis of a series of footsteps may comprise an analysis to look for missed footstep events and/or calculations of rhythm, hesitancy, and balance.

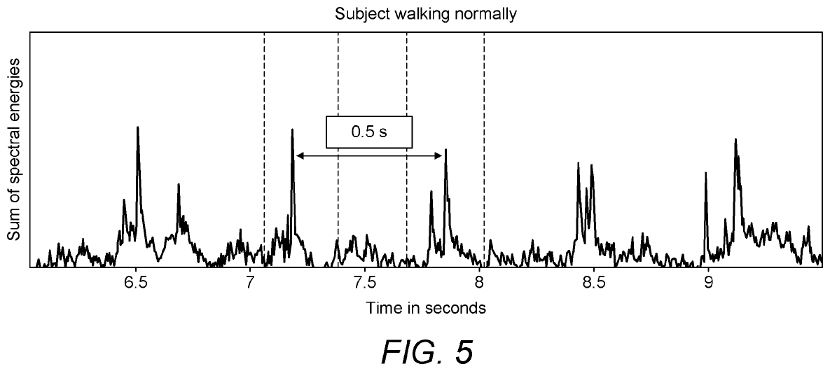

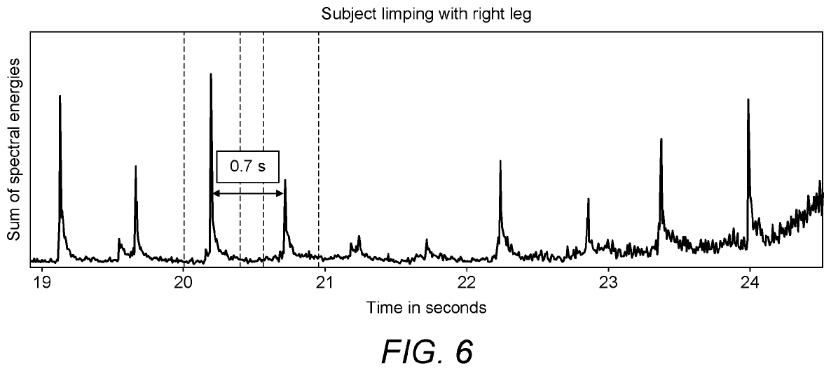

FIG. 6 shows an exemplary energy profile that can be expected when a subject is walking normally. In this case, the peaks in the energy profile and therefore the steps are separated by roughly 0.5 s. The footsteps shown in FIG. 5 are well balanced, as they peaks for each footstep are of similar amplitude.

The cadence (which may also be referred to as the rhythm) of a series of two or more footsteps may be calculated by determining the distance between peaks of the energy profile created by the sound of the footsteps. This may be calculated using beat analysis. Cadence is defined as a walking rate, which may be measured in steps per minute.

Similarly, the hesitancy in a series of two or more footsteps may be calculated by determining the distance (e.g. time) between a peak in the energy profile created by the sound of one footstep and a peak in the energy profile created by the sound of a subsequent footstep. The peaks may be corresponding peaks of the characteristic energy profile representing a footstep (e.g. as indicated in FIG. 5). From this, the footstep analysis unit 112 can derive a walking speed and stride length. The walking speed and stride length are indicative of the walking hesitancy of the subject—e.g. a low walking speed and/or a short stride length relative to a predefined walking speed and/or stride length for the height and/or leg/femur/inseam length of the subject are indicative of high hesitancy, and a high walking speed and/or a long stride length are indicative of low hesitancy. The walking speed and the stride length may be combined into a measure of hesitancy.

The balance of a subject taking the two or more footsteps may be determined by comparing the energy profile of left and right footsteps. Any imbalance in these energy profiles is indicative of an imbalance in the gait of the subject. Furthermore, if strides with one foot are longer than the strides with the other foot, this is indicative of an imbalance in the gait of the subject. A measure of balance may be, for example, a value representing a ratio of the height of one or more peaks in the characteristic energy profiles of left and right footsteps, or a ratio of the total energy of the characteristic energy profiles of left and right footsteps (e.g. the areas under the energy profiles of left and right footsteps). In some examples, a balance value of 1 may be indicative of balanced left and right footsteps; and a balance above or below 1 being indicative of an imbalance in favour of one or other of the left and right footsteps. One of more left footsteps may be combined to form an average left footstep for use in calculating a measure of balance; similarly, one of more right footsteps may be combined to form an average right footstep for use in calculating a measure of balance. FIG. 6 shows an exemplary energy profile that can be expected when a subject is walking with a limp. The amplitude of the second highlighted peak is significantly lower than the first highlighted peak.

The first neural network may comprise a deep neural network (DNN). More specifically, the first neural network may comprise a convolutional neural network (CNN). The first neural network may be trained by providing it with training data which has been analysed by other means (e.g. manually) so as to categorise each training example provided to the network (e.g. as being an example of normal, unbalanced, inconsistent, or hesitant footsteps). The training data may comprise balanced, non-hesitant footsteps with a consistent rhythm, and may also contain footsteps from subjects with impaired mobility having one or more of an unbalanced, hesitant, and inconsistent rhythm series of footsteps.

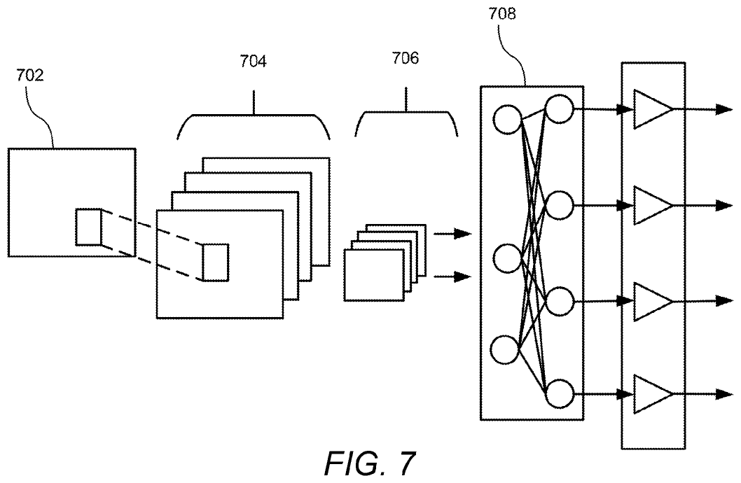

FIG. 7 shows an exemplary CNN used by footstep analysis unit 112. The input data in the time-frequency domain 702 is received by the footstep analysis unit. This data may be representable in the form of a spectrogram, i.e. a 2D array of signal energy plotted on time and frequency axes. At 704, convolution operation is performed on the data by applying weights to regions of the audio signal. After the convolution operation at 704 an activation function may be applied. The activation function may be one of a binary step function, a linear function, a sigmoid function, a Tanh function, a ReLU function, and a leaky ReLU function. After an activation function is applied, at 705 the CNN may perform a pooling operation to reduce the dimensions of the output of the convolution. The CNN may further comprise one or more fully connected layers.

The mobility factor determined using the first neural network is a measure of the mobility of the subject. The mobility factor may comprise the cadence, balance, and hesitancy (as described above) or combinations of two or more of these measures. The mobility factor may comprise one or more numerical values. The mobility of a subject may be classified in dependence on the mobility factor. For example, ranges may be defined for a numerical value of a mobility factor that indicate full mobility, partially impaired mobility, and significantly impaired mobility. If the mobility factor falls within a corresponding range, the mobility of the subject will be classified as full mobility, partially impaired mobility, and significantly impaired mobility respectively.

The mobility factor may be used to evaluate the stage of dementia for a subject. The severity of dementia is often assessed on a seven stage scale. Stages 1-3 usually indicate no dementia, stage 4 usually indicates early-stage dementia, stages 5-6 usually indicate mid-stage dementia, and stage 7 usually indicates late-stage dementia. Confusion and hesitation during walking results in measurable changes to the gait of a subject, which often occurs in mid-stage dementia. Hence, the present invention is able to distinguish between early and mid-stage dementia measuring differences in mobility.

Determining that two or more of the regions classified as containing the sound of a footstep correspond to a series of two or more consecutive footsteps of a subject may comprise identifying who generated the footstep sounds. However, this may not be necessary when the system is used in an environment where there is only one possible person walking in the vicinity of the microphone(s) 102. Identifying who generated a footstep sound may comprise using an unsupervised learning algorithm. More specifically, the unsupervised learning algorithm may comprise mixture model, for example a Gaussian mixture model (GMM). Unlike a supervised learning algorithm such as SVM, unsupervised learning algorithms do not need explicitly assigned labels to learn to classify new unseen data.

The unsupervised learning algorithm may be trained to identify the sound of the subject's footsteps. For example, footstep data for two subjects was obtained and two GMMs may be trained using said data, one GMM for each subject's footsteps. The GMM models may then be then incorporated into the logic of the footstep analysis unit 112. The footstep analysis unit 122 may receive a region of audio data containing footsteps, and then determine whose footsteps they are. Using this approach, the present invention has been shown to correctly identify the subject for 9 out of 10 footstep audio clips.

The system 100 may further comprise a trend analysis unit 114. The trend analysis unit 114 is configured to analyse the results of the footstep analysis unit 112 in order to determine changes in one or more of: spatiotemporal gait parameters like gait stride, gait velocity, gait cycle consistency and empirical health indicators derived from the former, like mobility and dynamic gait stability over longer periods of time (i.e. longer that a single series of steps). The trend analysis unit 114 may log each mobility factor determined by the footstep analysis, or it may log only a single mobility factor in a given time period and repeat this for several time periods, for example, by logging one mobility factor per day. The regularity at which mobility factors are logged by the trend analysis unit 114 may be predefined, dependent on detection of acoustic gait events by 112, or it may be adjustable by a user. The trend analysis unit 114 may be configured to use statistical models to track anomalous deviations in directly derived gait parameters like stride length or time and/or advanced machine learning and deep learning techniques to predict possibilities of anatomical issues and onset of illnesses marked by physiological symptoms. The trend analysis unit 114 may be configured to determine when the mobility factor increases or decreases by more than a predefined or adaptive threshold. The trend analysis unit 114 may also include aforementioned machine learning and deep learning software and/or hardware configured to detect physiological and anatomical manifestations of symptoms which can be studied and connected to either onset of illnesses like dementia, systemic fragility or higher probabilities of a fall. Significant changes in the mobility of a subject in terms of statistical moments like variance, kurtosis and/or skewness may be indicative of an increased risk of falling or of a worsening of a medical condition, for example dementia. The trend analysis unit 114 may be configured to use a statistical regression method to predict continuous gait patterns resembling falls or symptoms of medical illnesses. The trend analysis unit 114 may be configured to use a classification method to detect already available symptoms of medical illnesses or physiological abnormalities due to gait as a symptom and simulation methods (reinforcement learning and generative modelling) to predict future possible gait patterns from the current mobility and risk-of-fall state.

The system 100 may further comprise a back-end refinement unit 116. The refinement unit 116 may be configured to combine the quantitative results in the form of predictions of regression, classification or simulation-based techniques from footstep analysis unit 112 and trend analysis unit 114 for visual output for feedback unit 118. Refinement unit 116 may be utilized as a tool to evaluate, benchmark and compare results from footstep analysis unit 112 and trend analysis unit 114 before it is forwarded to the downstream components. Evaluation may include use of graphs/plots for internal monitoring of metrics like standard errors (absolute standard error, mean squared error, root mean squared error) for regression algorithms, multi-class and multi-label accuracy, F-score, recall (also called sensitivity), precision (also called specificity), Area Under Curve for Receiver Operating Characteristic (AUC-ROC), Polyphonic Sound Detection Score (PSDS) for classification algorithms, Dispersion across (DT), Short-Term Risk Across Time (SRT), Long-term Risk Across Time(LRT), Dispersion across Runs (DR), Risk Across Runs (RR), Dispersion across Fixed Policy Rollouts (DF), Risk across Fixed Policy Rollouts (RF) or any problem-specific reward function value that may have been defined for simulation based algorithms. Apart from evaluation metrics, graphs/plots may also be used to monitor feature separability of the various acoustic features that may be extracted via statistical or machine learning or deep learning techniques. Results from certain simulation based (reinforcement learning, generative learning and self-supervised based learning) algorithms may use simulation environments like Unity, Unreal Engine, PyBullet and its derivatives, Mujoco, OpenSIM and its derivatives, etc to visualize postures of human agents. These predicted postures can visualize a person (whose footsteps are being analysed in units 112 and 114) at high and low fall-risk probability scenarios. The simulation agents may be programmed to mimic and learn the gait pattern from an existing history of postures or real-time predicted postures of people and thereby extend the mimicking process indefinitely during varying simulated conditions (stimuli) when the input of postures is stopped. To generalize the predictions of unit 112 and unit 114, refinement unit 116 may include algorithms to map the learned mimicked gait pattern corresponding to varying body shapes (which may include dynamic and static alterations of height, weight, femur length, hip weight, size), specifically meaning to accommodate human bodies with abnormal gait or an underlying anatomical deformity or acquired condition.

The system 100 may further comprise a feedback unit 118. The feedback unit 118 may be configured to provide feedback indicative of one or of: the mobility factor (determined by the footstep analysis unit 112), and longer term changes/trends in mobility (determined by the trend analysis unit 114). The feedback unit 118 may be configured to provide feedback to one or more of the subject, a medical professional, and a designated person (for example a relative or a carer). The feedback unit 118 may be configured to provide one or more of visual, audible, and tactile feedback. Hence, the feedback unit 118 may comprises one or more lights or displays, one or more speakers, and/or one or more vibration systems. The feedback unit 118 may be configured to ask the subject to sit down, or to stand still. Standing still may allow the subject to regain proper balance and confidence to walk. The feedback unit 118 may be configured to assist the medical professional assess variations in gait by providing an indication of the parameters determined by trend analysis unit 114 over multiple periods of time and accept thresholding recommendations to relay to unit 114 to monitor for specific ranges of deviations in parameters logged by the latter. The feedback unit 116 may be configured to inform a relative or a carer designated to help and aid the subject about more immediate and/or short-term (daily or weekly observations) of anomalous results of trend analysis unit 114 which may include fall risk probabilities higher than a predefined and/or a manually defined threshold and sharp drops in mobility, stability and frailty parameters in gait.

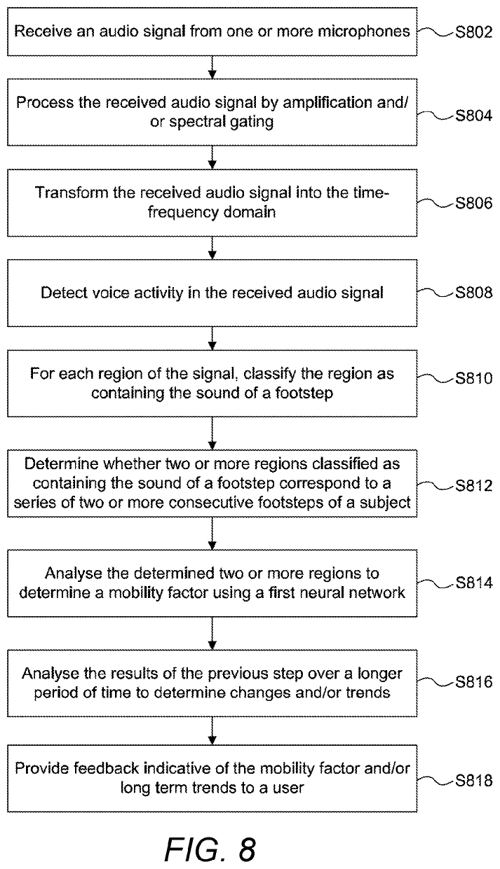

FIG. 8 shows an exemplary method of performing mobility analysis. At step S802, an audio signal is received from one or more microphones 102.

At step S802, the received audio signal may be amplified and/or spectrally gated. As described above, spectrally gating allows the relevant part of the received signal to be isolated and reduces the amount of data that needs to be processed downstream. Frequencies in the range of 10 Hz to 300 Hz may be isolated. As described above, this frequency range includes the characteristic signature of a footstep while excluding irrelevant frequencies.

At step S806, the signal may be transformed into the time-frequency domain. This may comprise performing a Fourier transform on the received audio signal.

At step 808, voice activity in the received may be detected. The audio signal may be stored in a memory in dependence on whether voice is detected in the received audio signal. For example, while the received audio signal contains a voice, the data may not be stored in memory. This provides additional privacy and data protection for users, particularly when monitory mobility in a home.

At step S810, for each of a plurality of overlapping regions of the audio signal, the signal region is classified as containing or not containing the sound of a footstep using a first supervised learning algorithm. The regions may comprise 1-2 s of the received audio signal. Each region may be delayed by 0.5-1 s compared to the preceding window.

At step S812, it is determined whether two or more of the regions classified as containing the sound of a footstep correspond to a series of two or more consecutive footsteps of a subject. This step may be performed using a neural network, for example in the manner described herein.

At step S814, the determined two or more regions are analysed to determine a mobility factor using a first neural network. This may comprise analysing the determined two or more regions to determine one or more of a cadence of the series of footsteps, a hesitancy of the subject, and a balance of the subject, and wherein the mobility factor is determined in dependence on one or more of the cadence, hesitancy, and balance.

The first neural network may be the same neural network used in step S812, or it may be a different neural network. Cadence is defined as a walking rate, which may be measure in steps per minute.

At step S816, the results of the previous step are analysed over a longer period of time to determine changes and/or trends. Changes in one or more of: spatiotemporal gait parameters like gait stride, gait velocity, gait cycle consistency and empirical health indicators derived from the former, like mobility and dynamic gait stability over longer periods of time (i.e. longer that a single series of steps) may be determined.

At step S818, feedback may be provided that is indicative of one or of: the mobility factor (determined by the footstep analysis unit 112), and longer term changes/trends in mobility (determined by the trend analysis unit 114). The feedback provided may be in the forms described above in respect of the feedback unit 118.

The system 100 and any unit comprising part of the system 100 may be any kind of device, machine or dedicated circuit, or collection or portion thereof, with processing capability such that it can execute instructions. A processor may be any kind of general purpose or dedicated processor, such as a CPU, GPU, system-on-chip, state machine, media processor, an application-specific integrated circuit, a programmable logic array, a field-programmable gate array (FPGA), or the like. A computer or computer system may comprise one or more processors. One or more of the units (for example the footstep analysis unit 114 and/or the trend analysis unit 116) may be located remotely from the microphone 102. For example, one or more of the units may comprise a server configured to receive the audio signal via an internet connection. The feedback unit 118 may be located remotely from the microphone(s) 102, such that the feedback is only provided to another person (for example a medical professional in a hospital) instead of the subject.

The methods described herein could be performed by one or more processors executing code that causes the processor(s) to perform the method. Examples of a computer-readable storage medium include a random access memory (RAM), read-only memory (ROM), an optical disc, flash memory, hard disk memory, and other memory devices that may use magnetic, optical, and other techniques to store instructions or other data and that can be accessed by a machine.

The terms computer program code and computer readable instructions as used herein refer to any kind of executable code for processors, including code expressed in a machine language, an interpreted language or a scripting language. Executable code includes binary code, machine code, bytecode, code defining an integrated circuit (such as a hardware description language or netlist), and code expressed in a programming language code such as C, Java or OpenCL. Executable code may be, for example, any kind of software, firmware, script, module or library which, when suitably executed, processed, interpreted, compiled, executed at a virtual machine or other software environment, cause a processor of the computer system at which the executable code is supported to perform the tasks specified by the code.

The applicant hereby discloses in isolation each individual feature described herein and any combination of two or more such features, to the extent that such features or combinations are capable of being carried out based on the present specification as a whole in the light of the common general knowledge of a person skilled in the art, irrespective of whether such features or combinations of features solve any problems disclosed herein, and without limitation to the scope of the claims. The applicant indicates that aspects of the present invention may consist of any such individual feature or combination of features. In view of the foregoing description it will be evident to a person skilled in the art that various modifications may be made within the scope of the invention.

What is claimed is:

1. A method for measuring the mobility of a subject, the method comprising:
receiving an audio signal from one or more microphones;
for each of a plurality of overlapping regions of the audio signal, classifying the region as containing the sound of a footstep using a first supervised learning algorithm;
determining that two or more of the regions classified as containing the sound of a footstep correspond to a series of two or more consecutive footsteps of a subject; and
using a first neural network, analysing the determined two or more regions to determine a mobility factor.

2. The method of claim 1, wherein analysing the determined two or more regions to determine a mobility factor using a first neural network comprises analysing the determined two or more regions to determine one or more of a cadence of the series of footsteps, a hesitancy of the subject, and a balance of the subject, and wherein the mobility factor is determined in dependence on one or more of the cadence, hesitancy, and balance.

3. The method of claim 1, wherein the first supervised learning algorithm comprises a support vector machine classifier.

4. The method of claim 1, wherein classifying a region as containing the sound of a footstep using the first supervised learning algorithm comprises:
analysing the region to locate one or more markers indicative of footstep events; and
if the region contains more than a predefined threshold number of footstep events, classifying that region as containing the sound of a footstep.

5. The method of claim 4, wherein classifying a region as containing the sound of a footstep using the first supervised learning algorithm comprises deriving a spectral energy of the region of the audio signal, wherein analysing the region to locate one or more markers indicative of footstep events comprises analysing the spectral energy of the region of the audio signal to locate one or more markers indicative of footstep events.

6. The method of claim 5, wherein classifying a region as containing the sound of a footstep using a first supervised learning algorithm comprises:
determining one or both of the mean and the variance of the spectral energy in the region of the audio signal; and
determining whether determined mean and/or variance of the spectral energy falls within a predefined range of an expected mean and/or variance respectively.

7. The method of claim 4, wherein a footstep event comprises one or more of: a heel strike, a tiptoe collision, and a toe scrape.

8. (canceled)

9. The method of claim 1, further comprising providing feedback indicative of the mobility factor to one or more users.

10. (canceled)

11. (canceled)

12. The method of claim 1, further comprising determining that a region classified as containing the sound of a footstep contains the sound of a footstep of the subject by applying an unsupervised learning algorithm trained to identify the sound of the subject's footsteps.

13. The method of claim 12, wherein the unsupervised learning algorithm comprises a Gaussian mixture model.

14. (canceled)

15. The method of claim 1, wherein the audio signal is received from two or more microphones.

16. The method of claim 15, further comprising capturing that part of the audio signal received by a first microphone and validating the captured audio signal by comparing the captured audio signal with that part of the audio signal received by a second microphone.

17. The method of claim 15, further comprising comparing the time that a feature in the audio signal is received by a first microphone with the time that a feature in the audio signal is received by a second microphone to determine a region of space from which the feature in the audio signal originated.

18. A system for measuring the mobility of a subject, comprising:
a footstep detection unit configured to receive an audio signal from one or more microphones and, for each of a plurality of overlapping regions of the audio signal, classify the region as containing the sound of a footstep using a first supervised learning algorithm; and
a footstep analysis unit configured to determine that two or more of the regions classified as containing the sound of a footstep correspond to a series of two or more consecutive footsteps of a subject and to, using a first neural network, analyse the determined two or more regions to determine a mobility factor.

19. The system of claim 18, wherein the footstep analysis unit is configured to analysing the determined two or more regions to determine a mobility factor using a first neural network by analysing the determined two or more regions to determine one or more of a cadence of the series of footsteps, a hesitancy of the subject, and a balance of the subject, and wherein the mobility factor is determined in dependence on one or more of the cadence, hesitancy, and balance.

20. The system of claim 18, wherein the first supervised learning algorithm comprises a support vector machine classifier.

21. The system of claim 18, wherein the footstep detection unit is configured to classify a region as containing the sound of a footstep using the first supervised learning algorithm by:
analysing the region to locate one or more markers indicative of footstep events; and
if the region contains more than a predefined threshold number of footstep events, classifying that region as containing the sound of a footstep.

22. The system of claim 21, wherein the footstep detection unit is configured to classify a region as containing the sound of a footstep using the first supervised learning algorithm by:
deriving a spectral energy of the region of the audio signal, wherein analysing the region to locate one or more markers indicative of footstep events comprises analysing the spectral energy of the region of the audio signal to locate one or more markers indicative of footstep events.

23. The system of claim 18, further comprising a feedback unit configured to provide feedback indicative of the mobility factor to one or more users.

24. (canceled)

25. A non-transitory computer readable storage medium having stored thereon computer readable instructions that, when executed at a computer system, cause the computer system to perform a method for measuring the mobility of a subject, the method comprising:
receiving an audio signal from one or more microphones;
for each of a plurality of overlapping regions of the audio signal, classifying the region as containing the sound of a footstep using a first supervised learning algorithm;
determining that two or more of the regions classified as containing the sound of a footstep correspond to a series of two or more consecutive footsteps of a subject; and
using a first neural network, analysing the determined two or more regions to determine a mobility factor.
